(19) United States
(12) Patent Application Publication
(10) Pub. No.: US 2017/0340233 A1
(43) Pub. Date: Nov. 30, 2017

(54) ELECTROCARDIOGRAM PATCH

(71) Applicant: Martin Kuster, Walchwil (CH)

(72) Inventor: Martin Kuster, Walchwil (CH)

(21) Appl. No.: 15/605,600

(22) Filed: May 25, 2017

Related U.S. Application Data
(60) Provisional application No. 62/341,344, filed on May 25, 2016.

(51) Int. Cl. A61B 5/0408; A61B 5/044; A61B 5/0432; A61B 5/00; A61B 5/04

(52) U.S. Cl. CPC A61B 5/04087; A61B 5/6823; A61B 5/0024; A61B 5/04012; A61B 5/0432; A61B 5/0006; A61B 5/044; A61B 2560/0443; A61B 2562/0209; A61B 2562/043; A61B 2562/046

(57) ABSTRACT

A device for measuring and recording body functions can include a first patch and a second patch. The first patch be applied to a user and include an electrocardiogram (“EKG”) sensor and a first communication circuit. The EKG sensor can be for recording electrical activity data of a heart of the user. The first communication circuit can be communicatively coupled to the EKG sensors for transmitting the electrical activity data. The second patch can be communicatively coupled to the first patch and include a processing device and a second communication circuit. The processing device can be for performing an analysis of the electrical activity data. The second communication circuit can be communicatively coupled to the first communication circuit for receiving the electrical activity data and communicatively coupled to the processing device for transmitting the analysis to a second device.

### CROSS REFERENCE TO RELATED APPLICATIONS

This disclosure is related to and claims the benefit of priority of U.S. Provisional Application No. 62/341,344, titled “Electrocardiogram Patch” and filed on May 25, 2016, which is hereby incorporated by this reference in its entirety.

### TECHNICAL FIELD

The present disclosure relates to a wearable medical device and, more particularly (although not necessarily exclusively), to an electrocardiogram patch.

### BACKGROUND

An electrocardiogram (“EKG”) is a test that can check for problems with a heart by recording electrical activity of the heart. The electrical activity of the heart can be recorded over a period of time using electrodes placed on a patient's body. These electrodes detect tiny electrical changes on the skin that arise from the heart muscle depolarizing during each heartbeat. Some EKGs use ten electrodes placed on the patient's limbs and the surface of the chest. The overall magnitude and direction of a heart's electrical depolarization can be captured at each moment through the cardiac cycle.

The electrodes are coupled to a device for processing the recorded electrical activity and for displaying the activity as a waveform. The waveform can be analyzed to help diagnose and treat health problems, as well as monitor the effectiveness of medication and implanted mechanical devices.

### SUMMARY

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various embodiments of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings and each claim.

According to certain embodiments of the present disclosure, a device can include a first patch and a second patch. The first patch be applied to a user and include an electrocardiogram (“EKG”) sensor and a first communication circuit. The EKG sensor can be for recording electrical activity data of a heart of the user. The first communication circuit can be communicatively coupled to the EKG sensors for transmitting the electrical activity data. The second patch can be communicatively coupled to the first patch and include a processing device and a second communication circuit. The processing device can be for performing an analysis of the electrical activity data. The second communication circuit can be communicatively coupled to the first communication circuit for receiving the electrical activity data and communicatively coupled to the processing device for transmitting the analysis to a second device.

According to certain embodiments of the present disclosure, a system can include a first patch, a second patch, and a display device. The first patch can be applied to a user and include a sensor and a first communication circuit. The sensor can be for recording bodily function data of the user. The first communication circuit can be communicatively coupled to the sensor for transmitting the bodily function data. The second patch can be communicatively coupled to the first patch and include a second communication circuit and a processing device. The second communication circuit can be communicatively coupled to the first communication circuit for receiving the bodily function data. The processing device can be for performing an analysis of the bodily function data. The display device can be communicatively coupled to the second patch for receiving the analysis from the processing device via the second communication circuit and displaying the analysis.

According to certain embodiments of the present disclosure, a method can include measuring, by an electrocardiogram (“EKG”) sensor, electrical activity data of a heart of a user. The EKG sensor can be coupled to a first patch applied to the user. The method can further include analyzing, by a processing device, the electrical activity data to determine an analysis of the electrical activity data. The processing device can be coupled to a second patch applied to the user. The method can further include transmitting, by a communication circuit, an analysis of the electrical activity data to a remote device.

Various implementations described in the present disclosure can include additional system, methods, features, and advantages, which can not necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

### BRIEF DESCRIPTION OF THE DRAWINGS

In the following detailed description, embodiments of the disclosure are described referring to the following figures:

FIG. 1 is a front view of a user with a main patch and an electrocardiogram patch according to one aspect of the present disclosure.

FIG. 2A is a front view of a main patch with a reusable module according to one aspect of the present disclosure.

FIG. 2B is a back view of a main patch with a reusable module according to one aspect of the present disclosure.

FIG. 3 is a block diagram of a main patch with a reusable module according to one aspect of the present disclosure.

FIG. 4 is a front view of an EKG patch with a reusable module according to one aspect of the present disclosure.

FIG. 5 is a block diagram of an EKG patch with a reusable module according to one aspect of the present disclosure.

### DETAILED DESCRIPTION

The subject matter of embodiments of the present invention is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

Certain aspects and features relate to a wearable device for measuring and recording body functions. The wearable device may record electrical data associated with the heart, skin temperature, heart rate, breathing intervals, and glucose level. The wearable device may also measure and record environmental factors such as acceleration, steps taken, and environmental temperature.

In some aspects, the wearable device can include one or more disposable patches. Some modules can be attached to a disposable patch and later removed from the patch such that the modules can be reused. The wearable device can be worn to provide constant measurement of a user's body functions, including the user's day-to-day life. In some aspects, the wearable device can be worn during sports, including extreme sports to monitor a player's health. In additional or alternative aspects, the wearable device can be a preparation tool for extreme activities, such as mountain climbing.

In some aspects, the device can be divided across two disposable patches; a main patch and an EKG patch. A main patch can contain sensors like skin temperature, heart rate, breathing intervals, and glucose levels, as well as environmental sensors like acceleration and steps. Some of the sensors can be coupled directly to one of the disposable patches for single use, or just for the lifetime of the disposable patch. For example, some sensors that need body contact may be for single use. Other sensors can be coupled to a reusable module that can be attached to one of the disposable patches. The sensors coupled to the reusable module can be removed from one disposable patch and reused by coupling the sensor to another disposable patch. The reusable module can also include a power supply, processing device, and communication unit. In some aspects, the power supply can be a rechargeable and exchangeable battery. In additional or alternative aspects, the communication unit can be a near field communication circuit that can power the wearable device based on received signals.

The main patch can be communicatively coupled to the EKG patch. The EKG patch can include a reusable module that can include a power supply and a communication unit. In some aspects, the reusable module can include a processing device. The EKG patch can also include sensors, such as EKG sensors for recording electrical activity of a heart. In some aspects, the EKG patch can be positioned on a user such that the EKG sensors are aligned with parts of the user's body. For example, a first sensor can be aligned with the fourth intercostal space to the right of the sternum, a second sensor can be aligned with the fourth intercostal space to the left of the sternum, a third sensor can be aligned midway between the second sensor and the fifth intercostal space at the midclavicular line, a fourth sensor can be aligned with the fifth intercostal space at the midclavicular line, a fifth sensor can be aligned with the anterior axillary line, and a sixth sensor can be aligned with the midaxillary line. In some aspects, the shape of the EKG patch and positioning of the EKG sensors may be based on a specific user or a group of users.

In some aspects, the main patch and the EKG patch can be communicatively coupled by a wire running between the two patches. In additional or alternative aspects, the patches can wirelessly communicate. For example, the patches may communicate using near field communication (“NFC”), a communication protocol that can enable wireless communication over short distances (e.g., within a few inches). In additional or alternative examples, the patches may implement NFC using electromagnetic induction between loop antennae. In some aspects, the body function data can be transmitted by the main patch to a second device. In some examples, the second device may display the body function data. In additional or alternative examples, the second device may analyze the body function data to determine if medical action should be taken.

These illustrative examples are given to introduce the reader to the general subject matter discussed here and are not intended to limit the scope of the disclosed concepts. The following sections describe various additional features and examples with reference to the drawings in which like numerals indicate like elements, and directional descriptions are used to describe the illustrative aspects but, like the illustrative aspects, should not be used to limit the present disclosure.

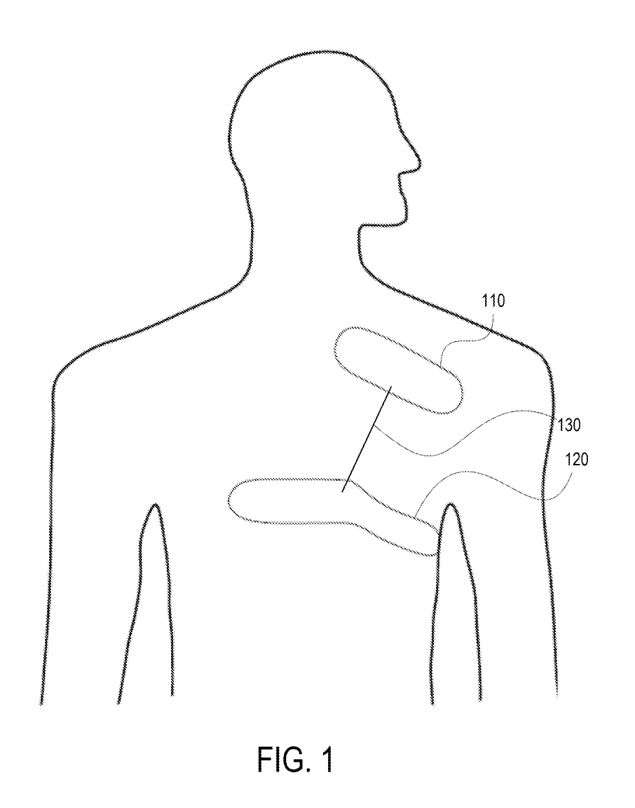

FIG. 1 is a front view of a user with a main patch 110 and an electrocardiogram (“EKG”) patch 120 according to one aspect of the present disclosure. In some aspects, the main patch 110 is communicatively coupled to the EKG patch 120 by a wire 130. In some examples, the wire 130 may be used to provide power from the main patch 110 to the EKG patch 120. In additional or alternative aspects, the main patch 110 can be wirelessly coupled to the EKG patch 120. In some example, the main patch 110 may communicate with the EKG patch 120 using NFC. In additional or alternative examples, the main patch 110 may provide power to the EKG patch 120 via a NFC signal.

Although the main patch 110 is described as providing power to the EKG patch 120, in some examples the EKG patch 120 may provide power to the main patch 110. In additional or alternative examples, at least one of the main patch 110 and the EKG patch 120 may receive power from an external device. For example, an external device using NFC may communicate with, and power, the main patch 110. In additional or alternative examples, the main patch 110 and EKG patch 120 may include batteries including reusable or exchangeable batteries. Although FIG. 1 illustrates a device with two separate patches, in some examples a device can include one or more than two disposable patches.

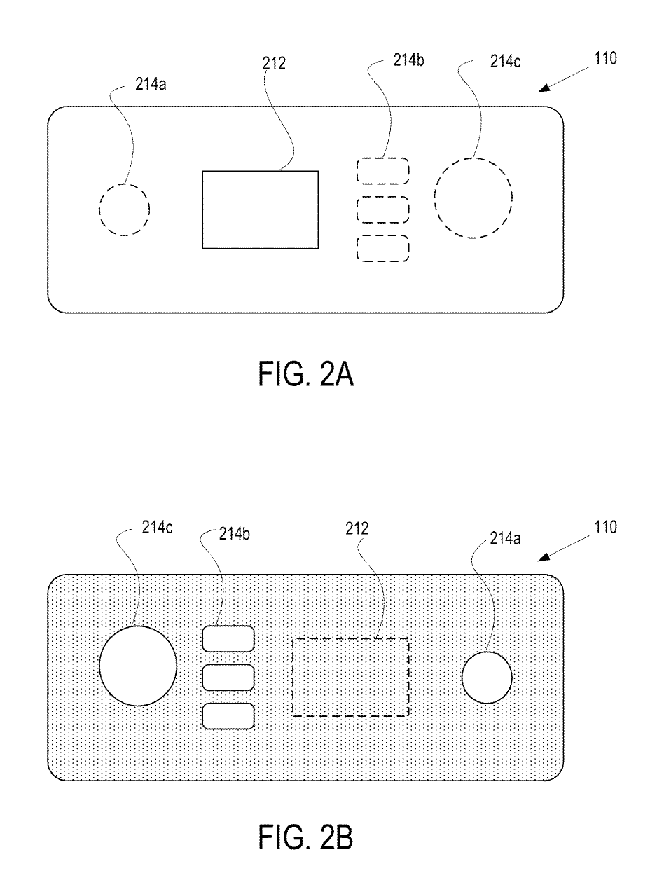

FIG. 2A is a front view of an example of the main patch 110 with a reusable module 212. In some aspects, the reusable module 212 can be detached from the main patch 110 and applied to a new main patch. The reusable module 212 can be attached to the main patch 110 using any suitable fastener or adhesive. For example, the reusable module 212 can be attached to the main patch 110 using tape. The reusable module 212 can include a processing device, a memory, a communication circuit, a power supply, and sensors. The main patch can further include sensors 214a-c coupled to a disposable portion of the main patch 110. The sensors 214a-c can be coupled to the front of the main patch 110 or embedded in the main patch 110. The sensors 214a-c can be isolated from the skin of the user and can measure environmental conditions.

FIG. 2B is a back view of the example of the main patch 110 with the reusable module 212. The back of the main patch 110 can include an adhesive for applying the main patch 110 to a user's skin. Some of the sensors 214a-c can be coupled to the back of the main patch 110 such that the sensors 214a-c can be applied directly to a skin of a user for measuring body functions. For example, the one or more sensors 214a-c can measure skin temperature, heart rate, breathing intervals, and glucose level. In some aspects, one or more sensors can be communicatively coupled to the reusable module 212 and can be reusable with different main patches.

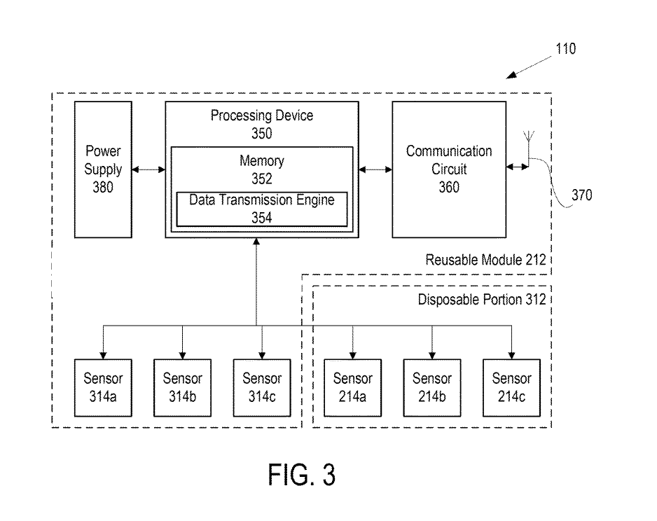

FIG. 3 is a block diagram of an example of the main patch 110 with the reusable module 212. The reusable module 212 includes a processing device 350, a communication circuit 360, an antenna 370, a power supply 380, and sensors 314a-c. The main patch 110 also includes a disposable portion 312, which includes the sensors 214a-c. The power supply 380 can be coupled to the processing device 350 for providing power to the processing device 350, which can provide power to the sensors 214a-c, 314a-c and communication circuit 360. In some aspects, the power supply 380 can be a rechargeable battery. In additional or alternative aspects, the power supply 380 can be an exchangeable battery. In additional or alternative aspects, the power supply 380 can be the communication circuit 360 using NFC to power the main patch 110 from received signals.

The communication circuit 360 can be communicatively coupled to the antenna 370 for wirelessly communicating with another device or another patch. In additional or alternative aspects, the communication circuit 360 can be communicatively coupled to another device or another patch (e.g., the EKG patch 120) by a wire (e.g., wire 130).

In some aspects, the sensors 214a-c, 314a-c can measure and record body functions such as skin temperature, heart rate, breathing intervals, and glucose level. In additional or alternative aspects, the sensors 214a-c, 314a-c can measure and record environmental factors of the user such as acceleration, steps taken, and environmental temperature. Some of the sensors 214a-c, 314a-c can be placed such that the sensors contact the body of a user. Other sensors 214a-c, 314a-c can be embedded within the main patch or coupled to the front side of the patch. In some aspects, the sensors 214a-c, 314a-c are communicatively coupled to the processing device 350 and can receive instructions to perform a measurement. In additional or alternative aspects, the sensors 214a-c, 314a-c constantly provide a stream of data to the processing device 350.

The processing device 350 can include any number of processors configured for executing program code stored in a memory 352. Examples of the processing device 350 can include a microprocessor, an application-specific integrated circuit (“ASIC”), a field-programmable gate array (“FPGA”), or other suitable processor. A combination of processing devices can be included in the main patch 110. For example, an ASIC may be included for performing preset functions and a FPGA may be included to allow additional functions to be added post manufacturing. In some aspects, the processing device 350 can be a dedicated processing device used for instructing the communication circuit 360 to transmit body function data. In other aspects, the processing device 350 can perform additional functions such as analyzing measurements from the sensors 214a-c, 314a-c.

The processing device 350 can include (or be communicatively coupled to) a non-transitory computer-readable memory. The memory 352 can include one or more memory devices that can store program instructions. The program instructions can include, for example, a data transmission engine 354 that is executable by the processing device 350 to perform certain operations described herein.

The operations can include receiving data from EKG sensors on an EKG patch (e.g., EKG patch 120 in FIG. 1). The operations can further include instructing the communication circuit 360 to transmit the data to a second device for displaying the EKG data as a waveform. The operations can further include instructing the communication circuit 360 to transmit additional body function data to the second device based on measurements from sensors 214a-c, 314a-c on the main patch 110.

Although FIG. 3 illustrates some of the sensors 214a-c on the reusable module 212 and some of the sensors 314a-c on the disposable portion 312, in some aspects all of the sensors 214a-c, 314a-c can be on the reusable module 212 or the disposable portion 312.

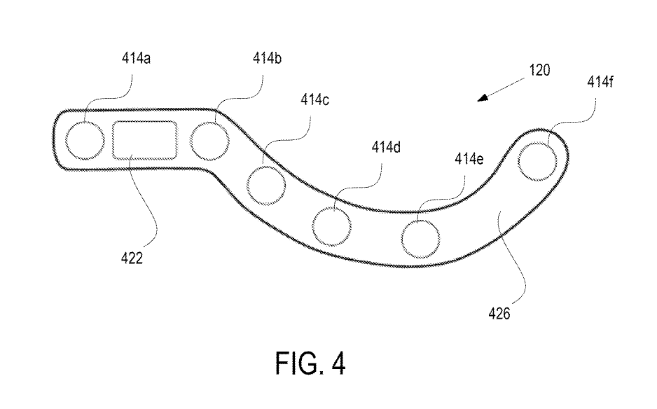

FIG. 4 is a front view of an example of the EKG patch 120 with a reusable EKG module 422. The EKG patch 120 can include one or more sensors; some sensors can be included on the reusable EKG module 422 and others sensors 414a-f can be included on the disposable portion 426. The sensors 414a-f can be for measuring electrical activity of the heart by detecting tiny electrical changes on the skin. The EKG patch 120 can be applied to a user's body using any adhesive. The shape of the EKG patch 120 and positioning of the sensors 414a-f on the patch can align the sensors 414a-f with regions of the user's body that facilitate a precise and accurate measurement of the electrical activity of the heart.

In some aspects, the reusable EKG module 422 can be detached for use with another EKG patch. In additional or alternative aspects, the reusable EKG module 422 can include components such as a processing device, power supply, and communication circuit for obtaining EKG data from the EKG sensors 414a-f and transmitting the EKG data to be analyzed.

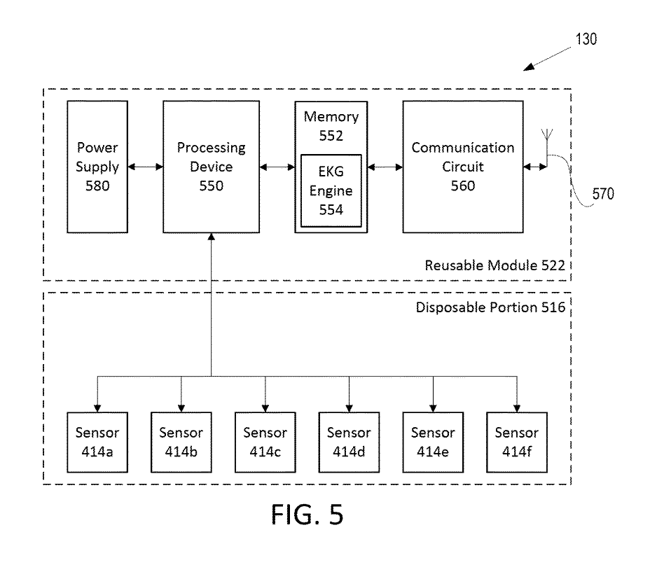

FIG. 5 is a block diagram of the EKG patch 120 with the reusable EKG module 422 according to one aspect of the present disclosure. The reusable EKG module 422 may be detached from the EKG patch 120 and applied to a new EKG patch. The reusable EKG module 422 includes a processing device 550, a memory 552, a communication circuit 560, an antenna 570, and a power supply 580. The disposable portion 516 can include the sensors 414a-f. The reusable EKG module 422 can also include one or more sensors (not depicted). In some aspects, the power supply 580 can be a rechargeable battery. In additional or alternative aspects, the power supply 580 can be an exchangeable battery. In additional or alternative aspects, the power supply 580 can be the communication circuit 560 using NFC to power the EKG patch 120 from received signals.

The communication circuit 560 can be communicatively coupled to the antenna 570 for wirelessly communicating with another device or another patch. In additional or alternative aspects, the communication circuit 560 can be communicatively coupled to another device or another patch (e.g., the main patch 110) by a wire (e.g., the wire 130).

In some aspects, the sensors 414a-f can measure and record body functions such as electrical activity of the heart, skin temperature, heart rate, breathing intervals, and glucose level. In additional or alternative aspects, the sensors 414a-f can measure and record environmental factors of the user such as acceleration, steps taken, and environmental temperature. Some of the sensors 414a-f can be placed such that the sensors 414a-f contact the body of a user. Other sensors 414a-f can be embedded within the EKG patch 120 or coupled to the front side of the EKG patch 120. In some aspects, the sensors 414a-f are communicatively coupled to the processing device 550 and receive instructions to take a measurement. In additional or alternative aspects, the sensors 414a-f constantly provide a stream of data to the processing device 550.

The processing device 550 can include any number of processors configured for executing program code stored in the memory 552. Examples of the processing device 550 can include a microprocessor, an ASIC, a FPGA, or other suitable processor. In some aspects, the processing device 550 can be a dedicated processing device. In other aspects, the processing device 550 can perform multiple functions.

The processing device can be communicatively coupled to (or include) a non-transitory computer-readable memory. The memory 552 can include one or more memory devices that can store program instructions. The program instructions can include for example, an EKG engine 554 that is executable by the processing device 550 to perform certain operations described herein. The operations can include receiving data from sensors 414a-f. The operations can further include instructing the communication circuit 560 to transmit the data to a main patch (e.g., the main patch 110) for analysis.

The foregoing description of certain examples, including illustrated examples, has been presented only for the purpose of illustration and description and is not intended to be exhaustive or to limit the disclosure to the precise forms disclosed. Numerous modifications, adaptations, and uses thereof will be apparent to those skilled in the art without departing from the scope of the disclosure.

What is claimed is:

1. A device comprising:
a first patch for application to a user comprising:
an electrocardiogram (“EKG”) sensor for recording electrical activity data of a heart of the user; and
a first communication circuit communicatively coupled to the EKG sensors for transmitting the electrical activity data; and
a second patch communicatively coupled to the first patch comprising:
a processing device for performing an analysis of the electrical activity data; and
a second communication circuit communicatively coupled to the processing device for transmitting the analysis to a second device, and communicatively coupled to the first communication circuit for receiving the electrical activity data.

2. The device of claim 1, wherein the EKG sensor comprises a plurality of EKG sensors positioned on the first patch for being aligned with portions of a body of the user.

3. The device of claim 2, wherein the plurality of EKG sensors comprises:
a first sensor positioned on the first patch for being aligned with a fourth intercostal space to the right of a sternum of the user;
a second sensor positioned on the first patch for being aligned with a fourth intercostal space to the left of the sternum of the user;
a third sensor positioned on the first patch for being aligned midway between the second sensor and a fifth intercostal space at a midclavicular line of the user;
a fourth sensor positioned on the first patch for being aligned with the fifth intercostal space at the midclavicular line of the user;
a fifth sensor positioned on the first patch for being aligned with an anterior axillary line of the user; and
a sixth sensor positioned on the first patch for being aligned with a midaxillary line of the user.

4. The device of claim 1, wherein the first patch further comprises a plurality of additional sensors for measuring additional data based associated with bodily functions or environmental conditions, the first communication circuit communicatively coupled to the plurality of additional sensor for transmitting the additional data to the processing device via the second communication circuit, the processing device for performing the analysis of the electrical activity data based on the additional data.

5. The device of claim 1, wherein the second patch further comprises a power supply for providing power to the processing device and the second communication circuit, the first communication circuit being a near field communication (“NFC”) circuit comprising an inductive loop for communicatively coupling to the second communication circuit, receiving NFC signals from the second communication circuit, and powering the EKG sensor based on the NFC signals.

6. The device of claim 1, wherein the first patch further comprises:
a disposable portion that includes an adhesive surface for attaching the first patch to the user; and
a reusable module including the EKG sensor, the reusable module being coupleable to the disposable portion such that the reusable module is decoupleable from the disposable portion and coupleable to another disposable portion of another device.

7. The device of claim 1, wherein the second patch further comprises:
a disposable portion that includes an adhesive surface for attaching the second patch to the user; and
a reusable module including the processing device, the reusable module being coupleable to the disposable portion such that the reusable module is decoupleable from the disposable portion and coupleable to another disposable module of another device.

8. The device of claim 1, wherein the second patch further comprises a memory device communicatively coupled to the processing device for storing the analysis during a first period of time in which the second communication circuit is beyond a communication range of the second device and providing the analysis to the second communication circuit during a second period of time in which the second communication circuit is within a range of the second device.

9. A system comprising:
a first patch for application to a user comprising:
a sensor for recording bodily function data of the user; and
a first communication circuit communicatively coupled to the sensor for transmitting the bodily function data;
a second patch communicatively coupled to the first patch comprising:
a second communication circuit communicatively coupled to the first communication circuit for receiving the bodily function data; and
a processing device for performing an analysis of the bodily function data; and
a display device communicatively coupled to the second patch for receiving the analysis from the processing device via the second communication circuit and displaying the analysis.

10. The system of claim 9, wherein the sensor comprises a plurality of EKG sensors positioned on the first patch for being aligned with portions of the user's body and for recording electrical activity data of a heart of the user, the plurality of EKG sensors comprising:
a first sensor positioned on the first patch for being aligned with a fourth intercostal space to the right of a sternum of the user;
a second sensor positioned on the first patch for being aligned with a fourth intercostal space to the left of the sternum of the user;
a third sensor positioned on the first patch for being aligned midway between the second sensor and a fifth intercostal space at a midclavicular line of the user;
a fourth sensor positioned on the first patch for being aligned with the fifth intercostal space at the midclavicular line of the user;
a fifth sensor positioned on the first patch for being aligned with an anterior axillary line of the user; and
a sixth sensor positioned on the first patch for being aligned with a midaxillary line of the user.

11. The system of claim 9, wherein the second patch further comprises a power supply for providing power to the processing device and the second communication circuit, the first communication circuit being a near field communication (“NFC”) circuit comprising an inductive loop for communicatively coupling to the second communication circuit, receiving NFC signals from the second communication circuit, and powering the sensor based on the NFC signals.

12. The system of claim 9, wherein the first patch further comprises:
a disposable portion that includes an adhesive surface for attaching the first patch to the user; and
a reusable module including the sensor, the reusable module being coupleable to the disposable portion such that the reusable module is decoupleable from the disposable portion and coupleable to another disposable portion of another device.

13. The system of claim 9, wherein the second patch further comprises:
a disposable portion that includes an adhesive surface for attaching the second patch to the user; and
a reusable module including the processing device, the reusable module being coupleable to the disposable portion such that the reusable module is decoupleable from the disposable portion and coupleable to another disposable portion of another device.

14. The system of claim 9, wherein the second patch further comprises a memory device communicatively coupled to the processing device for storing the analysis during a first period of time in which the second communication circuit is beyond a communication range of the second device and providing the analysis to the second communication circuit during a second period of time in which the second communication circuit is within a range of the second device.

15. A method comprising:
measuring, by an electrocardiogram (“EKG”) sensor, electrical activity data of a heart of a user, the EKG sensor coupled to a first patch applied to the user;
analyzing, by a processing device, the electrical activity data to determine an analysis of the electrical activity data, the processing device being coupled to a second patch applied to the user; and
transmitting, by a communication circuit, an analysis of the electrical activity data to a remote device.

16. The method of claim 15, further comprising:
storing, by the processing device, the analysis of the electrical activity data to a memory device coupled to the second patch;
determining, by the processing device, the remote device is within a communication range of the communication circuit; and
retrieving the analysis from the memory device based on the remote device being within the communication range of the communication circuit.

17. The method of claim 15, wherein the EKG sensor comprises a plurality of EKG sensors, wherein measuring electrical activity data of the heart of the user further comprises:
measuring a first portion of the electrical activity data by a first sensor of the plurality of EKG sensors positioned on the first patch aligned with a fourth intercostal space to the right of a sternum of the user;
measuring a second portion of the electrical activity data by a second sensor of the plurality of EKG sensors aligned with a fourth intercostal space to the left of the sternum of the user;
measuring a third portion of the electrical activity data by a third sensor of the plurality of EKG sensors aligned midway between the second sensor and a fifth intercostal space at a midclavicular line of the user;
measuring a fourth portion of the electrical activity data by a fourth sensor of the plurality of EKG sensors aligned with the fifth intercostal space at the midclavicular line of the user;
measuring a fifth portion of the electrical activity data by a fifth sensor of the plurality of EKG sensors aligned with an anterior axillary line of the user; and
measuring a sixth portion of the electrical activity data by a sixth sensor of the plurality of EKG sensors aligned with a midaxillary line of the user.

18. The method of claim 15, further comprising powering the EKG sensor based on a near field communication (“NFC”) signal received by a communication circuit conductively coupled to the EKG sensor and coupled to the first patch.

19. The method of claim 15, further comprising:
removing the first patch and the second patch from the user subsequent to transmitting the analysis of the electrical activity data to the remote device;
removing a first reusable module including the EKG sensor from the first patch;
removing a second reusable module including the processing device and the communication circuit from the second patch;
coupling the first reusable module to a third patch;
coupling the second reusable module to a fourth patch;
applying the third patch and the fourth patch to the user;
measuring, by the EKG sensor, additional electrical activity data of the heart of the user in response to applying the third patch including the first reusable module to the user;
analyzing, by the processing device, the additional electrical activity data to determine another analysis of the additional electrical activity data in response to applying the fourth patch including the second reusable module to the user; and
transmitting, by the communication circuit, the additional analysis of the electrical activity data to an additional remote device.

20. The method of claim 15, further comprising displaying, by the remote device, the analysis for use in diagnosing a health of the user.
